(19) United States
(12) Patent Application Publication
(10) Pub. No.: US 2005/0238706 A1
(43) Pub. Date: Oct. 27, 2005

(54) Pharmaceutically active lipid based formulation of SN-38

(75) Inventors: Imran Ahmad, Wadsworth, IL (US); Jia-Ai Zhang, Vernon Hills, IL (US)

Correspondence Address: LEYDIG, VOIT & MAYER, LTD., TWO PRUDENTIAL PLAZA, SUITE 4900, 180 NORTH STETSON, CHICAGO, IL, 60601-6780, US

(73) Assignee: NeoPharm, Inc., Lake Forest, IL (US)

(21) Appl. No.: 11/061,044

(22) Filed: Feb. 18, 2005

Related U.S. Application Data
(63) Continuation of application No. PCT/US03/25880, filed on Aug. 19, 2003.
(60) Provisional application No. 60/404,668, filed on Aug. 20, 2002.

(51) Int. Cl. A61K 9/127; A61K 31/4745

(52) U.S. Cl. 424/450; 514/283

(57) ABSTRACT

SN38, camptothecin derivatives are poorly water soluble, highly lipophilic camptothecin derivatives and are very active against a variety of human cancers. Because of their very poor water solubility, SN38 has not been used to treat human patients with cancer due to the inability to administer sufficient quantities of dissolved in a pharmaceutical formulation. This invention overcomes these limitations by teaching novel pharmaceutically acceptable SN38 liposome complex formulation for the direct administration of the formulation to human patients with cancer. The claimed invention also describes the methods to prepare liposomal SN38 complexes and antitumor compositions of liposomal SN38 complexes to allow the administration in sufficient amounts to treat various types of cancer and as antiviral agents. This invention is also directed to injectable sterile solutions, antitumor compositions, liposomes. The present invention is for novel compositions and methods for treating diseases caused by cellular proliferation, particularly, for treating cancer in mammals and more particularly in humans. The therapeutic compositions of the present invention include SN38 lipid complexes in which the complexes can contain any of a variety of neutral or charged lipids and, desirably, cardiolipin. The compositions are capable of efficiently incorporating SN38 into complexes and are capable of solubilizing relatively high concentrations of SN38.

### CROSS-REFERENCE TO RELATED APPLICATIONS

This application is a continuation of PCT/US03/25880, filed Aug. 19, 2003 which claims priority to U.S. Provisional Patent Application No. 60/404,668, filed Aug. 20, 2002. The disclosures of these applications are incorporated herein by reference thereto.

### FIELD OF THE INVENTION

This invention pertains to complexes of SN38 with lipids, their methods of manufacture, and their use as antiviral agents and in the treatment of diseases, especially diseases involving eukaryotic cellular proliferation.

### DESCRIPTION OF THE BACKGROUND

The compound known as 7-ethyl-10-hydroxycamptothecin (SN38) and more formally as ((+)-(4S)-4,11-diethyl-4,9-dihydroxy-1H-pyrano[3′,4′:6,7]-indolizino[1,2-b]quinoline-3,14(4H,12H)-dione, first disclosed in U.S. Pat. No. 4,473,692, is an active metabolite of irinotecan, a derivative of camptothecin. It is thought to bind to the enzyme topoisomerase I, the enzyme responsible for relieving torsional strain in DNA by inducing reversible single-strand breaks. The bound SN38 appears to block religation of the single-strand breaks by topoisomerase-I thereby causing cytotoxicity in mammalian cells which, apparently, can not otherwise sufficiently repair the breaks.

The metabolic conversion of irinotecan to SN38 occurs primarily in the liver by carboxylesterase-mediated cleavage of the carbamate bond between the camptothecin moiety and a dipiperidino side chain. Subsequently, this derivative undergoes conjugation to form the glucuronide metabolite.

SN38 is approximately 1000 times more potent than irinotecan as an inhibitor of topoisomerase I purified from human and rodent tumor cell lines. In vitro cytotoxicity assays show that SN38 is up to 2,000-fold more potent than irinotecan. Consequently, SN38 has the potential to be a highly effective antineoplastic agent. In addition, SN38 has an advantage over its camptothecin precursors in that it does not require activation by the liver. Therefore, an appropriate formulation could be used in local as well as systemic treatment methods.

SN38 is exceedingly insoluble in aqueous solutions. Despite its lack of solubility in water, it also has a low affinity for lipid membranes from which it tends to precipitate into aqueous phase. These solubility characteristics interfere with the use of SN38 as a therapeutic. Moreover, the effectiveness of SN38 after repeated administrations can be limited by the development of multi-drug resistance which not only reduces its effectiveness but also reduces the effectiveness of certain other antineoplastic therapeutics. The general toxicity of SN38 also limits its use therapeutically.

Thus, formulations are needed that improve SN38 efficacy such that SN38 can be used effectively in the treatment of diseases associated with cellular proliferation. Such a formulation should have suitable solubility and toxicity characteristics and will be useful as an antiviral agents and in the treatment of certain proliferative diseases such as cancer.

The invention provides such a composition and methods. These and other advantages of the present invention, as well as additional inventive features, will be apparent from the description of the invention provided herein.

### SUMMARY OF THE INVENTION

The present invention is for novel SN38 compositions, their preparation methods, and their use as antiviral agents and in treating diseases caused by proliferating eukaryotic cells, such as cancer, particularly in mammals, especially humans. The SN38 compositions include SN38 complexed with a lipid wherein more than 40 wt. % of the SN38 is complexed with the lipid. The complexes, include liposomes, and can contain any of a variety of neutral or charged lipid materials and, desirably, cardiolipin. Suitable lipids include any pharmaceutically acceptable lipophilic materials that bind SN38 to provide a stable pharmaceutical formulation and facilitate its administration to mammals. Cardiolipin can be synthetic, derived from natural sources, or be chemically modified. The lipid complexes can carry net negative, or positive charges, or can be neutral. Preferred complexes also contain α-tocopherol. The SN38 complexes can be used advantageously with secondary therapeutic agents other than the SN38 complexes, including antineoplastic (such as cisplatin, taxol, doxorubicin, vinca alkaloids, and temozolomide), antifungal, antibiotic, antiviral, and antimetabolites, or other active agents. Liposome complexes can be multilamellar vesicles, unilamellar vesicles, or their mixtures, as desired. The invention also encompasses methods for preparing such SN38 complexes. The invention is further directed to methods in which a therapeutically effective amount of the SN38 complexes are included in a pharmaceutically acceptable excipient and administered to a mammal, such as a human, as an antiviral agent or to treat proliferative diseases, such as cancer.

This invention also pertains to the methods to prolong shelf-life of SN38 complexes.

In one particularly preferred method of preparing the SN38 complexes, SN38 is dissolved in an alkaline solution and used to hydrate a lipid film to form liposomes.

### DETAILED DESCRIPTION OF THE PREFERRED EMBODIMENTS

The present invention provides compositions and methods for delivering SN38 to a mammalian host. The compositions and methods are characterized by avoidance of solubility problems of SN38, high SN38 and complex stability, ability to administer SN38 as a bolus or short infusion in a high concentration, reduced SN38 toxicity, increased therapeutic efficacy of SN38, and modulation of multidrug resistance.

The inventive composition is a lipid complex with SN38 in which the complex desirably contains cardiolipin. Suitable complexes are characterized by having SN38 bound with a lipophilic compound that imparts solubility characteristics such that stable pharmaceutical preparations can be generated and used. The complexes include, but are not limited to, liposomes, emulsions, and micelles. In the complexes the SN38 can be bound to the lipid by covalent, hydrophobic, electrostatic, hydrogen, or other bonds and is considered bound even where the SN38 is simply be entrapped within the interior of a liposome. The SN38 compositions include SN38 complexed with a lipid wherein at least about 40% or more, such as at least about 50 wt. % or more of the SN38 is complexed with the lipid, more preferably at least about 70 wt. % or more, even more preferably at least about 80 wt. % or more (e.g., at least about 85% or more), and most preferably at least about 90 wt. % or more (such as at least about 95% or more) of the SN38 is complexed with lipid (e.g., at least a portion of the lipid). Where the compositions are liposomal, desirably, at least about 70 wt. % or more, even more preferably at least about 80 wt. % or more (e.g., at least about 85% or more), and most preferably at least about 90 wt. % or more (such as at least about 95% or more) of the SN38 is entrapped or encapsulated with the liposomes.

Desirably, the SN38 lipid complexes contain cardiolipin. Any suitable cardiolipin can be used. For example, cardiolipin can be purified from natural sources or can be chemically synthesized or chemically modified, such as tetramyristylcardiolipin, by such methods as are known in the art.

SN38 complexes generally contain other complexing agents in addition to cardiolipin. Suitable agents include pharmaceutically acceptable synthetic, semi-synthetic (modified natural) or naturally occurring compounds having a hydrophilic region and a hydrophobic region. Such compounds include amphiphilic molecules which can have net positive, negative, or neutral charges or which are devoid of charge. Suitable complexing agents include compounds, such as phospholipids that can be synthetic or derived from natural sources, such as egg or soy. Suitable phospholipids include compounds such as phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM), and the like, alone or in combination. Phosphatidylglycerols such as dimyristoylphosphatidylglycerol, dioleoylphosphatidylglycerol, distearoylphosphatidylglycerol, dipalmitoylphosphatidylglycerol, diarachidonoylphosphatidylglycerol, are preferred, as are mixtures thereof. The phospholipids dimyristoylphosphatidylcholine (DMPC), dimyristoylphosphatidylglycerol (DMPG), dioleoylphosphatidylglycerol (DOPG), distearoylphosphatidyl choline (DSPC), dioleoylphosphatidylcholine (DOPC), dipalmitoylphosphatidylcholine (DPPC), diarachidonoyl phosphatidylcholine (DAPC), egg phosphatidylcholine, soy phosphatidylcholine, or hydrogenated soy phosphatidylcholine (HSPC) can be used, as can mixtures thereof. Other lipids that can be employed include ganglioside GM1 and polymer modified lipids, such as PEG modified lipids.

The SN38 lipid complexes generally include at least one sterol or steroid component such as cholesterol, polyethylene glycol derivatives of cholesterol (PEG-cholesterols), coprostanol, cholestanol, or cholestane, or α-tocopherol. They may also contain sterol derivatives such as cholesterol hemisuccinate (CHS), cholesterol sulfate, and the like. Organic acid derivatives of tocopherols, such as α-tocopherol hemisuccinate (THS), can also be used. Suitable SN38 complexes can also be formed with glycolipids, or natural or derivatized fatty acids and the like. The preferred SN38 complexing agents include cardiolipin (e.g., natural cardiolipin or synthetic cardiolipin), a phosphatidyl choline, cholesterol, and α-tocopherol which are combined to form of a liposome.

Any suitable amount of SN38 can be used. Suitable amounts of SN38 are those amounts that can be stably incorporated into the complexes of the present invention. The SN38 should preferably be present in the abovementioned compositions at a concentration of about 0.01 mg/ml to about 20 mg/ml, such as between about 0.1 mg/ml and about 20 mg/ml or between about 0.01 mg/ml and about 5 mg/ml, more preferably about 0.1 to about 4 mg/ml, still more preferably about 0.5 to 3 mg/ml, and even more preferably about 0.8 to 2, such as from about 1 or more to about 1.5 mg/ml. Suitable compositions also generally contain from about 1 to about 50 wt. % cardiolipin, or preferably about 2 to about 25 wt. % cardiolipin, or more preferably about 5 wt. % to about 20 wt. % cardiolipin. Such compositions also generally contain about 1 wt. % to about 95 wt. % phosphatidylcholine, or more preferably about 20 wt. % to about 75 wt. % phosphatidylcholine. The preferred compositions also generally contain α-tocopherol in a concentration of about 0.001 wt. % to about 5 wt. %.

The complexing agents can also be considered liposome-forming materials when they are used to generate liposomes by methods such as are known. To generate the desired complexes, they can be dissolved by themselves or with the other lipophilic ingredients, including SN38, in suitable solvents. Suitable solvents are those which provide sufficient solubility and can be evaporated without leaving a pharmaceutically unacceptable amount of a pharmaceutically unacceptable residue. For example, the cardiolipin can be dissolved in non-polar or slightly polar solvent such as ethanol, methanol, chloroform, methylene chloride, or acetone. SN38 also can be dissolved in an aqueous, typically alkaline, buffer (e.g., sodium carbonate, sodium bicarbonate, sodium hydroxide, sodium phosphate, sodium acetate, sodium citrate, calcium hydroxide, sodium biphosphate, ammonium acetate, Tris (hydroxy-methyl) aminomethane, sodium benzoate, and the like). The aqueous of SN38 can then be added to the lipid film and the resulting mixture vigorously homogenized to produce liposomes, emulsions and micelles, as desired.

The invention further provides a method for forming a lipid composition comprising SN38 or a compound in equilibrium with SN38. SN38 can be said to be stable as long as most of the drug retains its chemical structure or a chemical structure that is in equilibrium with its chemical structure. Chemical structures in equilibrium with SN38 specifically include structures that impart greater solubility at high pH but which are converted to SN38 when the pH is lowered.

Generally, the method involves mixing dissolved lipophilic ingredients together and evaporating or lyophilizing the solvent(s) to form a (preferably homogeneous) lipid phase or lipid film. The lipid phase can be formed, for example, in a suitable organic solvent, such as is commonly employed in the art. The lipid phase then is hydrated with a first aqueous solution including the SN38 (or a compound in equilibrium with SN38) so as to form lipid composition including the compound. Thereafter, the pH of the composition is reduced so as to convert some or all of the compound in equilibrium with SN38 to SN38.

Preferably, the lipid phase is a lipid film, which can be generated by known methods. For example, solvent evaporation can be accomplished by any suitable means that preserves the stability of the components. SN38 complexes, including liposomes or micelles, can then be formed by hydrating the lipid phase, such as by adding a suitable solvent to the dry lipid film mixture. Suitable solvents include pharmaceutically acceptable polar solvents. Generally, solvents are aqueous solutions containing pharmaceutically acceptable salts, buffers, or their mixtures. In one method, a lipid film is hydrated with an aqueous solution of SN38 having an alkaline pH. Suitable pHs range from about 7 to about 11, pHs of about 8 to about 10 are more preferred, and pHs of about 9 to about 10 are most preferred. Aqueous solutions having a suitable pH can be prepared from water having an appropriate amount of NaOH dissolved therein. Alternatively, such solutions can be prepared with buffers, such as Tris-HCl, which have pKs within about 1 pH unit of the desired pH. Other suitable buffers include sodium carbonate, sodium bicarbonate, sodium hydroxide, sodium phosphate, ammonium acetate, sodium citrate, sodium hydroxide, calcium hydroxide, sodium biphosphate, sodium phosphate, Tris (hydroxy-methyl) aminomethane, sodium benzoate, and the like.

Liposome complexes can be formed (during the hydration step, for example) by dispersing the lipid in the aqueous solution with vigorous mixing. Any method of mixing can be used provided that the chosen method induces sufficient shearing forces between the lipid film and polar solvent to strongly homogenize the mixture and form the desired complexes. For example, mixing can be by vortexing, magnetic stirring, and/or sonicating. Where multilamellar liposomes are desired, they can be formed simply by vortexing the solution. Where unilamellar liposomes are desired, a sonication or filtration step is included in the process.

Liposomal SN38 complexes can be prepared by mixing SN38, cardiolipin, cholesterol, phosphatidyl choline and α-tocopherol in a suitable solvent to form a homogeneous mixture. The mixture is dried to form a lipid film and hydrated into liposomes by the addition of water or an aqueous solution and mixing. Alternatively, SN38 liposomes can be prepared by dissolving the lipophilic ingredients (with the exception of SN38) together and evaporating them to form a lipid film. A solution of SN38 is prepared in an aqueous solution at alkaline pH then is used to hydrate the dry lipid film and form liposomes.

Alternatively, SN38 can be directly dissolved in alkaline aqueous buffer solution, such as previous described. The dissolved SN38 can be added to the liposomes that are prepared by any of the techniques now known or subsequently developed for preparing liposomes. For example, the liposomes can be formed by the conventional technique for preparing multilamellar liposomes (MLVs), that is, by depositing one or more selected lipids on the inside walls of a suitable vessel by dissolving the lipids in chloroform and then evaporating the chloroform, adding the aqueous solution which is to be encapsulated to the vessel, allowing the aqueous solution to hydrate the lipid, and swirling or vortexing the resulting lipid suspension to produce the desired liposomes.

Alternatively, techniques used for producing large unilamellar liposomes (LUVs), such as, reverse-phase evaporation, solvent dilution procedures, infuision procedures, and detergent dilution, can be used to produce the liposomes. A review of these and other methods for producing liposomes can be found in the text Liposomes, Marc J. Ostro, ed., Marcel Dekker, Inc., New York, 1983, Chapter 1.

In general, any suitable method of forming liposomes can be used so long as it generates liposome entrapped SN38. Multilamellar vesicles, stable plurilamellar vesicles, and reverse phase evaporation vesicles can be used. As can be appreciated, the present invention is intended to cover SN38-entrapped liposome compositions, however made.

Suitable liposomes can be neutral, negatively, or positively charged, the charge being a function of the charge of the liposome components and pH of the liposome solution. For example, at neutral pH, positively charged liposomes can be formed from a mixture of phosphatidyl choline, cholesterol and stearyl amine. Negatively charged liposomes can be formed, for example, from phosphatidyl choline, cholesterol, and phosphatidyl serine.

After formation of the lipid composition comprising SN38 or a compound in equilibrium with SN38, the pH of the composition is reduced so as to convert some or all of the compound in equilibrium with SN38 to SN38. Desirably, the pH of the composition is less than about 3.5 (e.g., a pH of from about 1 and 3.5, such as between about 1.5 and about 3), and preferably the pH is about 2.0. The pH can be reduced, in accordance with the inventive method, directly after the hydration stage, e.g., by adding an acidic buffer (such as those described herein), or after a step of dehydration (or drying), storage (if desired), and re-hydration (also termed “resuspension” or “reconstitution”), as described herein. Alternatively, the pH can be reduced during the re-hydration of a dried or lyophilized preparation, for example, where an acidic buffer is employed to reconstitute dried liposomes containing SN38.

Targeting agents can be bound to the SN38 complexes such that the complexes can be targeted to particular tissues or organs. The agents can be bound through covalent, electrostatic, or hydrophobic bonds with the complexes. Suitable targeting agents include carbohydrates and proteins (e.g., antibodies, antibody fragments, peptides, peptide hormones, receptor ligands, and mixtures thereof) or other agents as are known to target desired tissues or organs. For example, U.S. Pat. No. 6,056,973, which is herein incorporated by reference, discloses a number of targeting agents and target cells. (See col. 11, 1.1-41). Methods of preparing suitable conjugates are also disclosed. (See Col. 11,155-col. 14, 1.20).

SN38 complexes can be filtered through suitable filters to control their size distribution. Suitable filters include those that can be used to obtain the desired size range of liposomes from a filtrate. Accordingly, the liposomes produced are preferably treated to reduce their size and to produce a homogeneous population. This may be accomplished by conventional techniques such as extrusion through a filter, preferably of 100 to 800 nm pore size, the filter being either the straight path or tortuous path type. The filter preferably has a pore size of about 5 microns or less, and more preferably about 1 micron or less, such as about 500 nm or less, or even about 200 nm or less or 100 nm or less. Other methods of size reducing the liposomes to a homogenous size distribution are ultrasonic exposure, the French press technique, hydrodynamic shearing, homogenization using, for example, a homogenizer or microfluidization techniques. Alternatively, filtration can occur after formulation in liquid excipients or diluents, as hereinafter described.

Thus, for example, the liposomes can have a diameter (e.g., average mean diameter) of about 5 microns or less, and more preferably, about 1 micron or less, such as about 500 nm or less, or even about 200 nm or less or 100 nm or less. It is preferred that the liposomes used in the present invention have an average mean diameter from about 20 nm to about 1000 nm and preferably of from about 100 nm to about 800 nm or from about 100 nm to about 400 nm. An average mean diameter of about 160 nm is particularly preferred.

To improve shelf-life, the present invention provides SN38 liposome preparations which can be stored for extended periods of time without substantial leakage from the liposomes of internally encapsulated materials. The present invention provides SN38 liposome preparations which can be dried or dehydrated to form a dried lipid composition, stored for extended periods of time while dehydrated, and then rehydrated when and where they are to be used, without losing a substantial portion of loaded SN38 during the dehydration, storage and rehydration processes. The drying or dehydration can be achieved either after or before the pH of the composition is reduced.

The liposomes are preferably dried or dehydrated to form a dried lipid composition using standard freeze-drying equipment or equivalent apparatus, that is, they are preferably dehydrated under reduced pressure. If desired, the liposomes and their surrounding medium can be frozen in liquid nitrogen before being dehydrated. Alternatively, the liposomes can also be dehydrated without prior freezing, by simply being placed under reduced pressure.

To achieve these and other objects, the invention, in accordance with one of its aspects, provides SN38 liposome preparations that have been dehydrated in the presence of one or more protective sugars. In certain preferred embodiments of the invention, the liposomes are dehydrated with the one or more sugars being present at both the inside and outside surfaces of the liposome membranes. In other preferred embodiments, the sugars are selected from the group consisting of trehalose, maltose, lactose, sucrose, glucose, and dextran, with the most preferred sugars from a performance point of view being trehalose and sucrose. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration. The dehydration is accomplished under vacuum and can take place either with or without prior freezing of the liposome preparation.

It has been found that inventive liposomes having a concentration gradient across their membranes can be dried or dehydrated (preferably in the presence of one or more sugars), subsequently rehydrated, and the concentration gradient then used to create a transmembrane potential which will load SN38 into the liposomes. Alternatively, the concentration gradient can be created after the liposomes have been dehydrated and rehydrated. Accordingly, the invention provides a method of loading liposomes with SN38 or a compound in equilibrium with SN38 involving preparing a preparation which includes liposomes, dehydrating the liposome preparation, rehydrating the dehydrated preparation, replacing the external medium surrounding the liposomes in the rehydrated preparation with a medium (such as an acidic buffer, suitable examples of which are discussed below), which produces an ion concentration gradient capable of generating a transmembrane potential having an orientation which will load SN38 or a compound in equilibrium with SN38 into the liposomes; and admixing SN38 or a compound in equilibrium with SN38 with the liposomes in their replaced external medium.

Dried, dehydrated, or lyophilized SN38 complex liposomes can be resuspended (i.e., reconstituted) into a suitable solution (typically an aqueous solution) by gentle swirling of the solution. The rehydration can be performed at room temperature or at other temperatures appropriate to the composition of the liposomes and their internal contents. When desired, liposomes can be dried such as by evaporation or lyophilization and the liposomes resuspended (i.e., reconstituted) in any desirable polar solvent. Where liposomes are formed as described herein by hydrating lipid films with alkaline, aqueous solvents containing SN38, it is desirable to use a low pH buffer, such as those descried herein, to resuspend (reconstitute) the dehydrated or lyophilized liposomes. Suitable solvents for resuspending (reconstituting) the liposomes include, for example, a buffered solution (typically an aqueous solution) having a pH of less than about 3.5 (e.g., a pH of from about 1 and 3.5, such as between about 1.5 and about 3), and preferably having a pH of about 2.0 (e.g., a lactate buffered solution having a pH of about 2.0). In such embodiments, the resuspension of the dehydrated lipid composition can effect the reduction of pH of the composition.

When the dehydrated or lyophilized liposomes are to be used, rehydration (or reconstitution) can be accomplished by adding an SN38 activating agent to close the lactone ring of SN38. In this sense, the SN38 and compound in equilibrium with SN38 becomes is released as active (pharmaceutically active) SN38. Accordingly, the invention provides a method of making SN38 complexes comprising formulating dehydrated or lyophilized complexes containing liposomes and SN38 or a compound in equilibrium with SN38, dissolving or resuspending the dehydrated or lyophilized complexes in an aqueous solution, and contacting the liposomes with a activating agent such that SN38 becomes active. The activating agent can be any acidic aqueous buffers, e.g., sodium citrate, citric acid, sodium acetate, acetic acid, ascorbic acid, sodium lactate, lactic acid, sodium tartrate, tartartic acid, sodium succinate, succinic acid, aspartic acid, hydrochloric acid, meleic acid, sodium carbonate, sodium sulfate, sulfuric acid, preferably, sodium lactate, sodium acetate, and the like. In some embodiments, it can be desirable to employ a solubilizing agent to increase the solubility of SN38 during formulation, such as an alkaline buffer, examples of which are discussed herein. Also, it can be desirable for one or more pharmaceutically acceptable excipients to be employed to increase the shelf-life of the composition. Suitable excipients for enhancing shelf life include, for example, protective sugars, as disclosed herein.

The inventive liposomal compositions desirably are stable for at least about 24 hours, and more preferably, they are stable for at least about 48 hours. Most preferably, the liposomal compositions containing SN38, or other lipid complexes of the present invention, are stable for at least about 72 hours. Stability can be assessed either over the time post-formulation or over the period post-reconstitution following drying or lyophilization. In this context, the stability of a liposomal composition of the present invention over time can be assessed, for example, by assaying the change in mean particle size over a 24, 48, or 72 hour period. Typically, stability is assessed after maintaining the composition at room temperature (e.g., about 25° C.) for the desired period of time, but other suitable temperatures can be employed. Desirably, when measured at 25° C., the mean particle size of the composition after 24, 48, or 72 hours post-formulation or post-reconstitution varies (e.g. is increased or decreased) by less than about 25% (more preferably, the size varies by less than about 20% or 15%, and most preferably by less than about 10% or less than about 5%) of that when the composition is initially formulated or reconstituted. Stability alternatively can be assessed by measuring the pH of the composition over the desired time frame. Desirably, the pH of the composition after 24, 48, or 72 hours post-formulation or post-reconstitution varies (e.g., either is increased or decreased) by at most about 0.5 pH units, and more preferably by at most about 0.4 pH units, from the pH of the composition when the composition is initially formulated or reconstituted. More, preferably, the pH of the composition after 24, 48, or 72 hours post-formulation or post-reconstitution varies by at most about 0.3 pH units from the pH of the composition when initially formulated or reconstituted, and even more preferably by at most about 0.2 pH units from the pH of the composition when initially formulated or reconstituted. Most preferably, the pH of the composition after 24, 48, or 72 hours post-formulation or post-reconstitution varies by at most about 0.1 pH unit from the pH of the composition when initially formulated or reconstituted. Another measurement of stability is the entrapment efficiency of SN38 within the composition, especially a liposomal composition. Desirably, the entrapment efficiency of SN38 within the composition after 24, 48, or 72 hours post-formulation or post-reconstitution is at least about 80% (more preferably at least about 85%, and most preferably at least about 90% or at least about 95%) of that when the composition is initially formulated or reconstituted. Most preferably, the entrapment efficiency of SN38 within the composition measured 24, 48 or 72 hours post formulation or reconstitution does not appreciably change from that measured when the composition is first formulated or reconstituted.

The invention includes pharmaceutical preparations, which, in addition to non-toxic, inert pharmaceutically suitable excipients, contain the SN38 complex and methods for preparing such compositions. By non-toxic, inert pharmaceutically suitable excipients there are to be understood solid, semi-solid or liquid diluents, fillers and formulation auxiliaries of all kinds.

The invention also includes pharmaceutical preparations in dosage units. This means that the preparations are in the form of individual parts, for example capsules, pills, suppositories and ampoules, of which the content of the SN38 complex corresponds to a fraction or a multiple of an individual dose. The dosage units can contain, for example, 1, 2, 3 or 4 individual doses or ½, ⅓ or ¼ of an individual dose. An individual dose preferably contains the amount of SN38 which is given in one administration and which usually corresponds to a whole, a half, a third, or a quarter of a daily dose.

Tablets, dragees, capsules, pills, granules, suppositories, solutions, suspensions and emulsions, pastes, ointments, gels, creams, lotions, powders and sprays can be suitable pharmaceutical preparations.

For the oral mode of administration, the SN38 complex can be used in the form of tablets, capsules, losenges, powders, syrups, aqueous solutions, suspensions, and the like. Carriers such as lactose, sodium citrate, and salts of phosphoric acid can be used to prepare tablets. Further, disintegrants such as starch, and lubricating agents, such as magnesium stearate, sodium lauryl sulfate and talc can be included. Diluents such as lactose and high molecular weight polyethylene glycols can be used in the preparation of dosages in capsule form. The active ingredient can be combined with emulsifying and suspending agents to generate aqueous suspensions for oral use. Flavoring agents such as sweeteners can be added, as desired.

For topical administration and suppositories drug complexes can be provided in the form of such gels, oils, and emulsions as are known by the addition of suitable water-soluble or water-insoluble excipients, for example polyethylene glycols, certain fats, and esters or mixtures of these substances. Suitable excipients are those in which the drug complexes are sufficiently stable to allow for therapeutic use.

The abovementioned pharmaceutical compositions are prepared for administration in the usual manner according to known methods, for example by mixing the complexed SN38 with suitable excipient(s).

The present invention also includes the use of SN38 according to the invention and of pharmaceutical preparations which contain SN38 according to the invention in human and veterinary medicine for the prevention, amelioration and/or cure of diseases, in particular those diseases caused by cellular proliferation, such as cancer, in any mammal, such as a cow, horse, pig, dog or cat. However, it is particularly preferred for use in the treatment of human patients, particularly for cancer and other diseases caused by cellular proliferation. In a preferred embodiment, the inventive method is employed to treat a disease caused by disease caused by proliferating eukaryotic cells in a patient homozygous for the wild-type UGTA1 allele or having at least one copy of a mutant UGTA1 allele (i.e., heterozugous or homozygous), such as, for example, UGTA1*28. Patients having mutations in UGTA1 can exhibit impaired capacity for glucoronidation of SN38; accordingly, employing the inventive compositions, such as the inventive liposomal formulations, can improve efficacy in such patients. The inventive compositions have particular use in treating human cancers and viral infections, in addition to multiple sclerosis. Example of cancers treatable by this invention include, but not limited to lung cancer (such as non-small cell lung cancer); breast cancer; testicular cancer; ovarian cancer; gastro intestinal cancers including colon, rectal, pancreatic, and gastric cancers, hepatocellular carcinoma; head and neck cancers; prostate cancer; renal cell carcinoma; adenocarcinoma; sarcomas; lymphomas; leukemias; and mycosis fugoides; melanoma; high grade glioma, glioblastoma and brain cancers.

The inventive complxes including SN38 (or a compound in equilibrium with SN38) also can be employed to treat viral infections within a patient. In this regard, the invention provides a method of treating viral infections comprising administering to a patient having a viral infection composition comprising a complex comprising SN38 or a chemical in equilibrium with SN38 and a lipid to the patient in an amount sufficient to treat the viral infection within the patient. The application of the inventive method can treat a viral infection by mediating the symptoms of the infection or, in some patients, by killing infected cells or decreasing the viral load within said patient. The method can be employed to treat infections by many viruses, such as adenoviruses, herpes viruses, papillomaviruses, pox viruses, SARS viruses, and immunodeficiency viruses. A preferred viral infection that can be treated in accordance with the inventive method include immunodeficiency viruses, such as SIV, FIV, and, most preferably, HIV.

The active compound or its pharmaceutical preparations can be administered dermally, orally, parenterally, intraperitoneally, intravenously, rectally, or directly to a tumor (e.g., via intratumoral injection). As SN38 does not require activation by the liver, it is advantageous to employ the present compositions locally, such as by directed injection into an arm or leg, or in the case of a human, a hand or a brain.

In a human of about 70 kg body weight, for example, about 0.1 to 2 mg or about 0.5 to 1 mg SN38 can be administered per kg of body weight can be administered. Preferably, about 0.5 to 2.0 mg of SN38 per kg of body weight is administered. Dosing also can be calculated per body surface area, and, for human patients, it is preferred to administer the inventive composition in amounts of from about 2 mg/m² to about 150 mg/m² or to deliver a dose of SN38 of such amounts. More preferably, between about 2 or about 2.5 mg/m² and about 125 mg/m² of the composition, such as between about 2.5 mg/m² and about 30 mg/m² (e.g., about 2.5 mg/m², about 5 mg/m², about 10 mg/m², about 20 mg/m², or about 25 mg/m²), is administered to a patient, or an amount of the composition is administered to deliver such dosage of SN38 to the patient. Also, dosing of about 30 mg/m², about 40 mg/m², about 50 mg/m², about 60 mg/m², about 70 mg/m², about 80 mg/m², about 90 mg/m², or about 100 mg/m² also is suitable. However, it can be necessary to deviate from the dosages mentioned and in particular to do so as a function of the nature and body weight of the subject to be treated, the nature and the severity of the illness, the nature of the preparation and if the administration of the medicine or other chemotherapeutic agent, and the time or interval over which the administration takes place. Thus it can suffice in some cases to manage with less that the abovementioned amount of active compound whilst in other cases the abovementioned amount of active compound must be exceeded.

For human patients, a preferred dosing regimen involves administration of the composition over a period of from about 30 or about 180 minutes, such as between about 60 and about 120 minutes, and more preferably for a period of about 90 minutes. Other dosing regimens and the type of administration of the SN38 can be determined by one skilled in the art, by available methods. Suitable amounts are therapeutically effective amounts that do not have excessive toxicity, as determined in empirical studies.

A significant advantage of cardiolipin-containing compositions is that they provide a method of modulating multidrug resistance in cancer cells which are subjected to SN38. In particular, the present compositions reduce the tendency of cancer cells subjected to chemotherapy with SN38 to develop resistance thereto, and reduces the tendency of cancer cells to develop resistance to other therapeutic agents, such as taxol or doxorubicin. Thus, other agents (e.g., secondary therapeutic agents) other than the SN38 complexes (such as the liposomal SN38 compositions) can be advantageously employed with the present treatment in combination with the SN38 complexes. Suitable adjunctive secondary therapeutic agents include, for example, antineoplastic agents (such as cisplatin, taxol, doxorubicin, vinca alkaloids, and temozolomide), antifungal agents, antibiotic agents, antiviral agents, antimetabolites, imunomodelators, and other secondary active agents. Preferred secondary agents include, for example, Gonadotropin release hormone, antiestrogens, antiandrogens, cisplatin, carboplatin, oxaliplatin, antisense oligonucleotides, paclitaxel, docetexl, vinca alkaloids, such as vincristin, vinblastine, vindestine and vinorelbine, doxorubincine, daunorubicin, epirubicin, mitoxantrone, cytarabine, temozolomide, leuprolide, cyclophosphamide, etoposide, and Tamoxifen, among other secondary agents.

Having described the present invention, reference will now be made to certain examples which are provided solely for purposes of illustration and which are not intended to be limiting.

### EXAMPLE 1

SN38 (3 μmoles) can be dissolved in chloroform containing 3 μmoles cardiolipin. To this mixture, 14 μmoles of phosphatidyl choline dissolved in hexane and 10 μmoles cholesterol in chloroform can be added. The mixture can be stirred gently and the solvents can be evaporated under vacuum at below 30° C. to form a thin dry film of lipid and drug. Liposomes can then be formed by adding 2.5 ml of saline solution and aggressively mixing the components by vortexing. The flasks can then be vortexed to provide multilamellar liposomes and optionally sonicated in a sonicator to provide small unilamellar liposomes. The efficiency of SN38 encapsulation can be determined by dialyzing an aliquot of the subject liposomes overnight in a suitable aqueous solvent or centrifuging an aliquot of the subject liposomes at 200,000×g. for 2 hour at 4° C. Thereafter the liposome fraction is dissolved in methanol and analyzed by standard methods using high pressure liquid chromatography (HPLC), such as reverse phase HPLC. Generally the encapsulation efficiency of SN38 in liposomes will be between 80 to 95% of the initial input dose.

### EXAMPLE 2

Similar experimental conditions can be utilized with varying quantities of drug and lipid. For example, concentrations of 6 μM SN38, 6 μM cardiolipin, 28 μM phosphatidyl choline and 20 μM cholesterol can be used by dissolving them in a suitable solvent, evaporating the solvent, and dispersing the dried lipid/drug film in a suitable aqueous solvent such as 5 ml of 7% trehalose-saline solution. Hydration of the liposomes can be facilitated by vortexing and/or sonicating the mixture. The liposomes can then be dialyzed, as desired, and the percent encapsulation of SN38 in liposomes measured, as described above. Typically, SN38 encapsulation will be greater than about 75% and more generally between about 85 to 95% or more as assayed by HPLC.

### EXAMPLE 3

SN38 can be encapsulated in liposomes by using 3 μM of the drug, 15 μM of dipalmitoyl phosphatidyl choline, 1 μM cardiolipin, and 9 μM cholesterol in a volume of 2.5 ml. The drug and lipid mixture can be evaporated under vacuum and resuspended in an equal volume of saline solution. The remainder of the process can be similar to that described above. The SN38 encapsulation efficiency will generally be higher than 75% in this system.

### EXAMPLE 4

In this example, liposomes containing 2 μM SN38, 2 μM of phosphatidyl serine, 11 μM phosphatidyl choline, 2 μM cardiolipin, and 7 μM cholesterol prepared by the method described in Example 1 is contemplated with greater than 75% SN38 encapsulation efficiency.

### EXAMPLE 5

In this example liposomes containing over 2 mg/ml SN38 in solution are demonstrated.

A lipid film is prepared by adding about 0.2 g of D-α-tocopherol acid succinate to about 1 kg of t-butyl alcohol which is warmed to about 35-40° C. The solution is mixed for about 5 min until the tocopherol is dissolved. About 6.0 g of tetramyristoyl cardiolipin is added to the solution and the solution is mixed for about 5 minutes. About 10 g of cholesterol is added to the solution and the solution is mixed for about 5 more minutes then about 30 g of egg phosphatidyl choline is added and mixed for another 5 min. Approximately 11 grams of the resulting lipid solution is lyophilized to generate a lipid film.

To prepare liposomal SN38, a 4 mg/ml solution of SN38 is prepared by dissolving the drug in an aqueous alkaline solution having a pH of between 8 and 10. Approximately 15 ml of this SN38 solution is added to a vial containing the lipid film. The vial is swirled gently, allowed to hydrate at room temperature for 30 min, vortexed vigorously for 2 min, and sonicated for 10 min in a bath-type sonicator at maximum intensity. The pH of the liposome solution is reduced to acid pH. Using this method more than 90 wt. % of the SN38 is complexed with lipid in the form of liposomes.

### EXAMPLE 6

Lipids, DOPC, cholesterol and cardiolipin at the appropriate ratios and tocopheryl acid succinate were dissolved in dichloromethane and subsequently dried under vacuum. The dried lipid film was rehydrated in the SN38 solution in 10% sucrose in 0.1N NaOH (pH>9). The lipid dispersion was extruded under nitrogen through 0.2 μM and 0.1 μM polycarbonate filters and then lyophilized to yield the LE-SN38 cake. The lyophilized cake was hydrated with 10 mM lactate buffer (pH 2.0) in order to convert the SN38 (open-lactone ring, inactive form) to the active form of the drug and allow its migration into the lipid bilayer. Analysis of the batch of reconstituted LE-SN38 showed 99.8% drug entrapment by ultracentrifugation and HPLC methods, stable entrapment upon dilution in normal saline and a mean vesicle size of 150 nm.

### EXAMPLE 7

Lipids were dissolved in ethanol. The lipid alcohol mixture was then dispersed in SN38/sucrose solution pH at 8-10. The bulk liposomal SN38 was then extruded through 0.2 μM and 0.1 μM polycarbonate filters. Following size-reduction, the product was then heated to 40° C. under vacuum to evaporate the organic solvent and then sterile filtered through 0.22 μM filters and lyophilized. The drug entrapment efficiency was >95% assay by HPLC method.

### EXAMPLE 8

A study was conducted to monitor the physical and chemical stability of LE-SN38 for up to 72 hours post-reconstitution. The objective of this study was to determine SN38 entrapment efficiency, SN38 concentration, liposome particle size, as well as pH at 25° C. over 72 hours post-reconstitution.

TABLE 1
Stability data for reconstituted liposomal SN38 at 25° C.

| Time/ | | | Entrapment | Mean | |
|---|---|---|---|---|---|
| Storage | | SN38 | Efficiency | Particle | |
| Condition | | % of initial | (%) | Size (nm) | pH |
| Initial | 25° C. | 100 | >95 | 258.8 | 2.60 |
| 0 hr | | | | | |
| 8 hr | 25° C. | 101 | >95 | 224.2 | 2.60 |
| 24 hr | 25° C. | 100 | >95 | 226.0 | 2.59 |
| 48 hr | 25° C. | 101 | >95 | 226.8 | 2.52 |
| 72 hr | 25° C. | 102 | >95 | 250.6 | 2.49 |

As seen in table 1, there does not appear to be any change in SN38 concentration at 25° C. condition over the course of the stability study as the % of initial SN38 concentration is found to be essentially 100% at all time points. In addition, the percent SN38 entrapment remains greater than 95% throughout the 72-hour study at 25° C. The mean vesicle diameter of reconstituted LE-SN38 at the initial time point is observed to be 258.8 nm. No drastic changes in particle size and pH were observed over 72 hour post-reconstitution. These results demonstrate that the inventive composition is stable over at least about 72 hours.

### EXAMPLE 9

A study was conducted to monitor the physical and chemical stability of LE-SN38 for up to 24 hours post-reconstitution and dilution in normal saline. The objective of this study was to determine particle size, SN38 entrapment efficiency, SN38 concentration, as well as pH at 25° C. as a function of time.

TABLE 2
Stability data for 8-fold diluted reconstituted
liposomal SN38 at 25° C.

| Time/ | | SN38 | | Mean | |
|---|---|---|---|---|---|
| Storage | | % of | SN38 | Vesicle | |
| Condition | | initial | Entrapment (%) | Size (nm) | pH |
| 0 hr | 25° C. | 100 | >95% | 173.5 | 2.98 |
| 8 hr | 25° C. | 99.2 | >95% | 183.0 | 2.95 |
| 24 hr | 25° C. | 98.7 | >95% | 193.4 | 2.96 |

As seen in table 2, there does not appear to be any significant change in SN38 concentration over the course of the stability study at 25° C. The percent SN38 entrapment remains greater than 95% throughout the 24-hour study at 25° C. No drastic changes in particle size and pH were observed over 24 hour study at 25° C. These results demonstrate that the inventive composition is stable over at least about 24 hours.

### EXAMPLE 10

A long-term stability study was conducted to monitor the physical and chemical stability of lyophilized LE-SN38 for up to 12 months. The objective of this study was to determine the visual appearance, particle size, SN38 entrapment efficiency, SN38 concentration, as well as pH at 25° C. as a function of time.

### Long-Term Stability Data for Lyophilized LE-SN38

TABLE 3
Lot# 1

| Time/ | | | | | | Mean |
|---|---|---|---|---|---|---|
| Storage | | SN38 | | | Entrapment | Particle |
| Condition | | % of initial | pH | Appearance | Efficiency (%) | Size (nm) |
| Initial | 2-8° C. | 100 | 2.89 | Off-white | >95% | 177.4 |
| 3 month | 2-8° C. | 102 | 2.51 | Off-white | >95% | 180.8 |
| 9 month | 2-8° C. | 99.1 | 2.50 | Off-white | >95% | 186.8 |
| 12 month | 2-8° C. | 102 | 2.54 | Off-white | >95% | 181.9 |

TABLE 4
Lot# 2

| Time/ | | | | | |
|---|---|---|---|---|---|
| Storage | | SN38 | | | Entrapment |
| Condition | | % of initial | pH | Appearance | Efficiency (%) |
| Initial | 2-8° C. | 100 | 2.73 | Off-white | >95% |
| 3 month | 2-8° C. | 100 | 2.45 | Off-white | >95% |
| 10 month | 2-8° C. | 97.0 | 2.46 | Off-white | >95% |
| 12 month | 2-8° C. | 99.6 | 2.50 | Off-white | >95% |

As seen in tables 3 and 4, the lyophilized LE-SN38 is stable up to 12 months. There are no significant changes in SN38 concentration, pH, drug entrapment and particle size up to 12 months.

### EXAMPLE 11

In-Vitro Cytotoxicity Study of LE-SN38

In vitro cytotoxicity of liposomal SN38 (LE-SN38) and CPT-11 in cancer cell lines was determined using Sulforhodamine B (SRB) assay (Monks, J Natl Cancer Inst, 83, 757-766 (1991)). A total of 8 cancer cell lines, including human colon cancer (HT29), human lung cancer (A549), human breast cancer (MX-1), human ovarian cancer (OVCAR-3), human pancreatic cancer (Capan-1), mouse Leukemia (P388), mouse adriamycin resistant leukemia (P388/ADR) and Lewis lung carcinoma (LLC), were included in this study. The G150 value was calculated as the concentration of LE-SN38 or CPT-11 that gives 50% growth inhibition.

Study showed that all eight cell lines studied were sensitive to LE-SN38 with GI₅₀ less than 0.1 μM. These results are comparable to previously reported data of free SN38 dissolved in DMSO (Lavelle et al., Semin Oncol, 23;1 Suppl 3, 11-20 (1996)); Cavaletti et al., Toxicol Lett, 118, 103-107 (2000)) indicating SN38 was released from the liposomes during the period of incubation of LE-SN38 in cell cultures and inhibited cell growth. Results showed that LE-SN38 was approximately 200 to 2000 fold more cytotoxic than CPT-11 against all tumor cell lines.

### EXAMPLE 12

Multiple Dose Toxicity Study Of LE-SN38 in CD2F1 Mice

CD2F1 mice (Male and Female) were obtained through Harlen Sprague Dawley Laboratories (Indianapolis, Ind.). The average weight of mice on day 1 of study was 16-22 g for females and 20-27 g for males, and the age was 6-7 weeks. Mice were pre-weighed individually prior to experiment. On days 1-5, animals were injected intravenously via tail vein with LE-SN38 or placebo liposomes at 5, 7.5 and 10 mg/kg dose levels. All animals were observed once daily during the study periods for mortality and clinical signs. Animals showing toxicity as manifested by clinical signs and body weight loss of 25% or more were considered as moribund and euthanized immediately.

The results of the multiple-dose toxicity study of LE-SN38 in CD2F1 mice indicated that the average weight loss ranged from 5.2% for 5 and 7.5 mg/kg dose groups (5 and 7.5 mg/kg×5 days) and 15.7% for 10 mg/kg dose group (10 mg/kg×5 days). However, the weight lost was recovered by day 17 post treatment for all LE-SN38 treatment groups. Animals in all groups were acting normal on day 1-5 post injection of LE-SN38. On day 6-12, animals treated with 5 and 7.5 mg/kg for 5 days were also normal, whereas animals treated with 10 mg/kg for 5 days showed clinical symptoms manifested by hunched posture, rough coat, dehydration and decreased activity. However, on day 14-18 post injection, all animals from all groups recovered. In general, LE-SN38 was well tolerated in mice at all dose levels studied. This could be attributed to the use of non-toxic lipids to form liposomes that buffered the toxicity of SN38. The retention of the drug in the liposomes reduced the tendency of SN38 molecules to directly interact with normal cells, therefore, attenuating the overall toxicity related to free SN38.

### EXAMPLE 13

Acute Dose and Multiple Dose Toxixcity of LE-SN38 in CD2F1 Mice: 30 Day Survival

CD2F1 mice (5-8 weeks of age), were obtained from Harlan Sprague Dawley Laboratories (Indianapolis, Ind.). Animals were housed in cages in temperature and humidity controlled room with 12 h light/dark cycles in animal care facility. Mice were offered ad libitum 8656 HT Rodent Diet (Harlan Teklad, Madison, Wis.). For acute dose toxicity study, LE-SN38 was intravenously (IV) administered to mice (two injections via tail vein/Day within 1 hour apart) at doses of 23, 28, 37, 46 and 65 mg/kg. For multiple dose toxicity study, mice were administered LE-SN38 (IV×5 days, once daily) at doses of 5.0, 7.5 and 10 mg/kg. The animals were observed for clinical signs of toxicity, mortality and body weight changes for up to 30 days.

The acute dose toxicity study suggested 37 and 46 mg/kg maximum tolerated dose (MTD) of LE-SN38 for male and female mice respectively. The MTD of LE-SN38 in a multiple dose toxicity study was found to be 5 and 7.5 mg/kg for male and female mice respectively. No significant loss of body weight was observed in mice at tolerated doses. In addition, no difference in hematological parameters were observed between control and drug treated groups. The results of these experiments are presented in tables 5 and 6.

TABLE 5
Acute Dose Toxicity of LE-SN38 in CD2F1 Mice: 30 day Survival

| | |
|---|---|
| Dose of LE-SN38 | Number of Mice Surviving/Total on Day 30 |

| (mg/kg) | Female | Male |
|---|---|---|
| 0 | 5/5 | 5/5 |
| 23 | 5/5 | 5/5 |
| 28 | N/A* | 5/5 |
| 37 | N/A | 5/5 |
| 46 | 5/5 | 0/5 |
| 65 | 4/5 | 0/5 |

CD2F1 mice were intravenously administered LE-SN38 (two injections via tail vein/day in 1hour apart). For 0 mg/dose, empty liposomes with a lipid amount of the highest dose group was used.
*N/A, not available.

TABLE 6
Multiple Dose Toxicity of LE-SN38 in CD2F1 Mice: 30 day Survival

| Dose of LE-SN38 | | |
|---|---|---|
| (mg/kg, once daily × 5 | Number of Mice Surviving/Total on Day 30 | |
| days) | Female | Male |
| 0 | 10/10 | 10/10 |
| 5 | 10/10 | 10/10 |
| 7.5 | 10/10 | 9/10 |
| 10 | 9/10 | 7/10 |

CD2F1 mice were administered LE-SN38 (iv, once daily × 5 days) with doses of 5.0, 7.5 and 10 mg/kg LE-SN38. For 0 mg/dose, empty liposome with a lipid of highest dose group was used.

### EXAMPLE 14

Therapeutic Efficacy of LE-SN38 and CPT-11 in Xenograft Mouse or Models

Either female CD2F1 (6-8 weeks old) mice or female C.B-17 SCID mice (4-6 weeks old) were obtained from the vendor and maintained as described previously. The CD2F1 mice were transplanted with P388 murine leukemia tumor cells, whereas the SCID mice were transplanted with HT-29 human colon cancer cells, Capan-1 human pancreatic cancer cells and MX-1 human breast cancer cells. After a suitable waiting period (waiting period varied based on the tumor models), each mouse received intravenous injection via tail vein of placebo liposomes, LE-SN38 or CPT-11 at pre-determined dose levels. For P388 bearing mice, the long term survival for each treatment group was assessed, whereas for solid tumor bearing mice, the tumor growth inhibition of placebo liposomes, LE-SN38 or CPT-11 at different dose levels was measured after 28 day post treatment.

Table 7 summarizes the therapeutic efficacy of LE-SN38 and CPT-11 against different tumors in mice. For the P388 tumor bearing mice administered with CPT-11 at doses of 4, 8 and 16 mg/kg for 5 consecutive days, the median survival time was 16, 20 and 22 days, respectively with no long-term survival. About 22% long-term survival (60 days) was observed for the mice administered with 16 mg/kg CPT-11. In contrast, when the mice were given LE-SN38 at doses of 2.76 mg/kg and 5.52 mg/kg for 5 consecutive days, 60% and 100% long-term survival (60 days) were observed at the respective dose level. There were no clinical signs of toxicity, such as diarrhea, hunched posture, scruffy fur and alopecia or weight loss at these dose levels of LE-SN38. Evidently, LE-SN38 exhibited significantly greater therapeutic efficacy against P388 murine leukemia tumor than the prodrug CPT-11.

When LE-SN38 was given to the mice bearing HT-29 human colon tumor at dose 2, 4 and 8 mg/kg, LE-SN38 inhibited human colon cancer growth by 46, 70 and 88%, respectively at 28 days post treatment. However, when the mice were treated with CPT-11 at the same dose levels, only 36% inhibition was observed at the highest dose level (8 mg/kg). At 2 and 4 mg/kg dose levels, CPT-11 did not show any inhibition against tumor growth. Clearly, LE-SN38 exhibited much greater inhibition against HT-29 induced tumor in mice than the prodrug CPT-11 at the same dose level.

Additionally, LE-SN38 exhibited greater growth inhibition against Capan-1 human pancreatic tumor growth in the animal groups treated with LE-SN38 than those treated with CPT-11 (Table 7). It was demonstrated that the antitumor efficacy of LE-SN38 against human pancreatic tumor in SCID ectopic model was superior to CPT-11. Moreover, it was also found that LE-SN38 induced a dose-dependent tumor regression of MX-1 human breast solid tumor in SCID mice. When the mice were treated with LE-SN38 at 4 and 8 mg/kg dose levels, the tumor regressed by 43.9% and 87.8% respectively. However, when the mice were given CPT-11 at 8 mg/kg dose level, no significant reduction of tumor size was observed.

It is known that intravenous administration of liposomes will lead to their accumulation in extravascular sites that exhibit leaky vasculature, as in the case for the tumor site. The extent of this accumulation could lead to an increase in tissue specific delivery of SN38 corresponding to several orders of magnitude greater than its precursor, CPT-11. This passive delivery of drug to sites of therapeutic activity may be accounted for the better efficacy of SN38 versus CPT-11. Liposomes also protect SN38 from structural transformation and/or chemical degradation. This protection of the active molecule could also have led to a significant increase in bioavailability, which ultimately enhanced the drug potency and efficacy. In summary, the antitumor efficacy of LE-SN38 was much greater than that of CPT-11 at the same dose levels.

TABLE 7
The Results of Multiple-Dose Therapeutic Efficacy Studies of LE-SN38 in Comparison with CPT-11

| | Tumor Models | | | |
|---|---|---|---|---|
| | P388 | HT-29 | Capan-1 | MX-1 |
| | Murine Leukemia | Human Colon | Human Pancreatic | Human Breast |
| | 60-day % Survival | % Growth Inhibition^{a} | % Growth Inhibition^{a} | % Growth Inhibition^{a} |
| Treatment | (n = 10) | (n = 5) | (n = 5) | (n = 5) |
| Placebo liposomes | 0 | 0 | 0 | 0 |
| CPT-11 | | | | |
| 2 mg/kg × 5 | n/t | 0 | n/t | n/t |
| 4 mg/kg × 5 | 0 | 0 | n/t | n/t |
| 8 mg/kg × 5 | 0 | 36 | 47.8 | 0 |
| 16 mg/kg × 5 | 22 | n/t | 68.7 | n/t |
| LE-SN38 | | | | |
| 2 mg/kg × 5 | 60 (2.78 mg/kg × 5) | 46 | n/t | n/t |
| 4 mg/kg × 5 | 100 (5.52 mg/kg × 5) | 70 | 60.4 | 43.9 |
| 8 mg/kg × 5 | n/t | 88 | 97.8 | 87.8 |
| 12 mg/kg × 5 | n/t | n/t | 98.0 | n/t |

n/t: not tested
^{a}% Growth Inhibition is defined as the percentage of final tumor volume
as compared to the initial tumor volume. It is calculated using the
following formula:
$\% Growth Inhibition = \frac{{V}_{1}}{{V}_{initial}} \times 100$
The drug treatment was initiated when the tumor reached to a size of
65-120 mm³.
The final tumor was measured on day 28 post treatment. The number of
mice used in each treatment for the studies ranged from 5-10.

### EXAMPLE 15

SN38 liposome complexes were prepared using the following procedure: the lipids were mixed with cardiolipin. The mixed powdered lipids were dissolved in chloroform in a round bottomed flask. The clear solution was placed on a Buchi rotary evaporator at 30° C. for 30 min to make a thin film. The flask containing the thin lipid film was dried under vacuum for 30 min. The film was hydrated in SN38 alkaline solution containing sucrose. The hydrated lipid film was rotated in a 50° C. The mixture in the flask was votexed and mixed. The mixture was sequentially extruded through decreasing size filters: 800 nm, 400 nm, 200 nm, and 100 nm. The SN38 liposome complexes were then lyophilized under vacuum. The resulting dehydrated complexes can be stored at 2-8° C. for at least 12 months. Prior to administration, the SN38 can be activated by adding acidic buffer.

### EXAMPLE 16

This example demonstrates the use of the inventive liposomal SN38 (LE-SN38) formulations in the treatment of patients with advanced cancer.

A study was conducted to assess the maximum tolerated dose and dose limiting toxicity of liposomal SN38, to determine the pharmacokinetics of SN38 after administration of LE-SN38, and to observe antitumor effects of LE-SN38.

The LE-SN38 was prepared by reconstitution with 5 mL of 10 mM lactate buffer and was stable for up to 8 hours refrigerated at 2-8° C. or at room temperature, 20-25° C. After dilution with normal saline, LE-SN38 was administered intravenously over 90 minutes on day 1 of a 21 day cycle. The first cycle consisted of a pre-dose, 15 & 45 min after infusion start, end-of-infusion; and a post-infusion at 5, 15 & 30 min; 1, 2, 3, 4, 6, 8, 12 & 24 h; 2, 4, 7, 14 & 21 days.

Patients involved in the study were individuals with advanced solid tumors who had failed conventional therapy. These consisted of three strata according to genotype:
Stratum A: Patients with UGT1A1 wt allele (homozygous)
Stratum B: Patients with UGT1A1*28 allele (heterozygous)
Stratum C: Patients with UGT1A1*28 allele (homozygous)
Dosages of 2.5 mg/m², 5 mg/m², and 10 mg/m² and 20 mg/m² were employed in this study. A dose level LE-SN38 had to be tolerated by Stratum A patients before enrollment began at that dose level for Stratum B patients, and a dose level LE-SN38 had to be tolerated by Stratum B patients before enrollment began at that dose level for Stratum C patients. There were between 3 and 6 patients/cohort/strata. Total plasma SN38 concentration and plasma SN38-glucoronide concentration were assessed for each patient.

From this study, it was observed that LE-SN38 was well tolerated when given up to 20 mg/m² in Stratum A patients, and LE-SN38 was well tolerated at lower doses when given in the first cohorts to Stratum B Patients. From the study, it was observed that the pharmacokinetics of SN38 is proportional to dose from 2.5 to 10 mg/m². Also, no difference was observed in the pharmacokinetics of SN38 of Strata A and Strata B cancer patients given the 2.5 mg/m² LE-SN38.

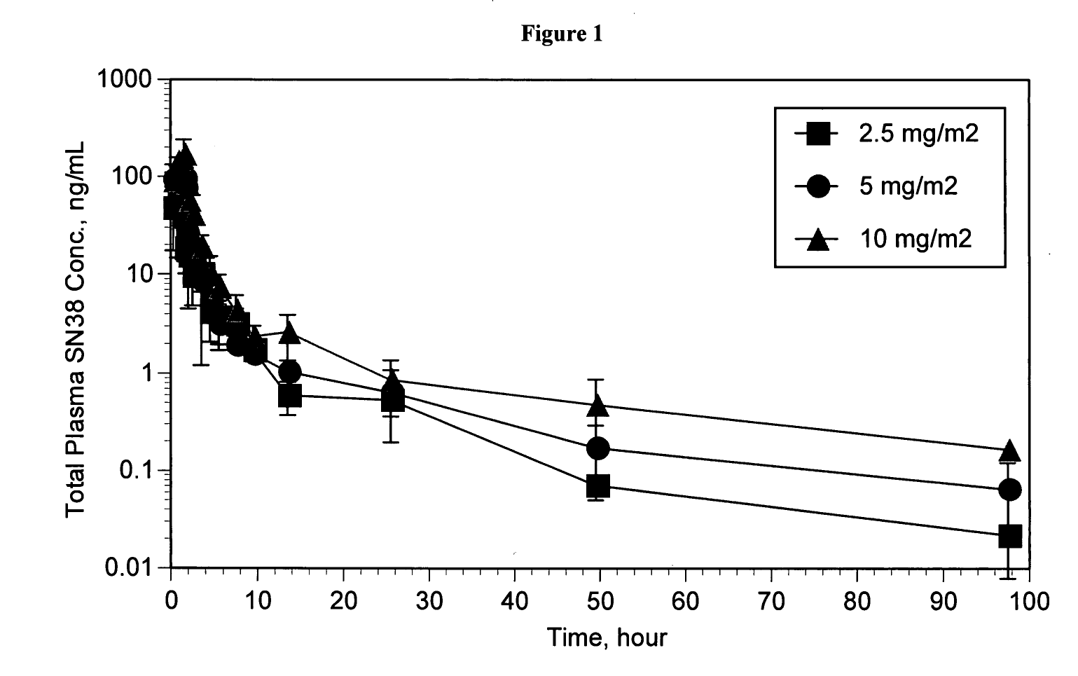

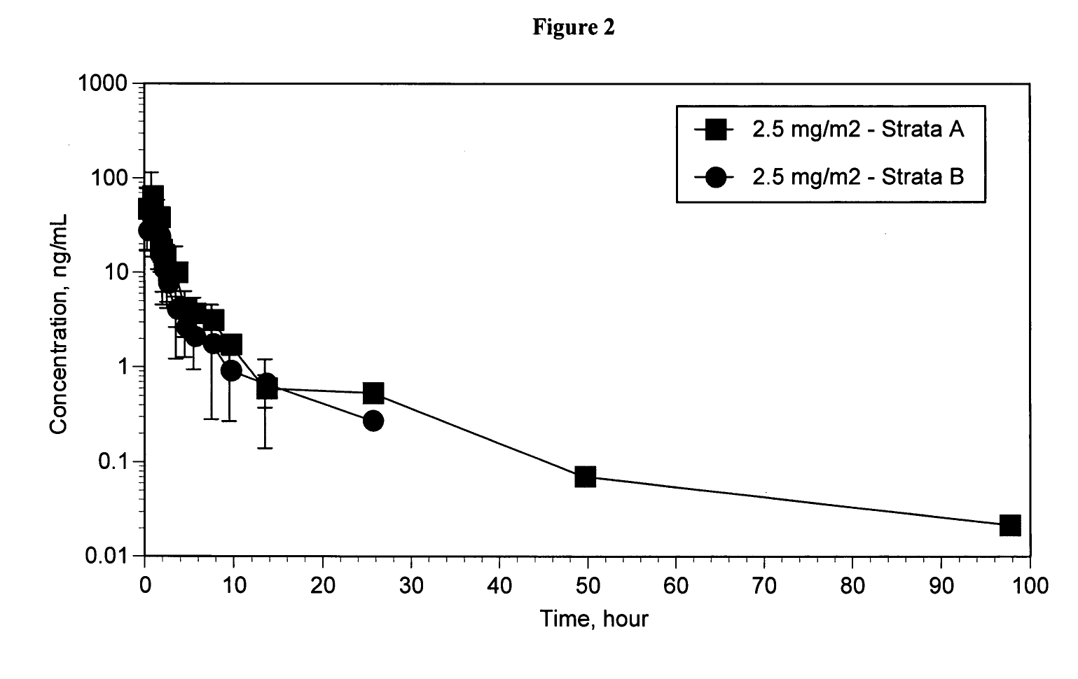

Table 8 presents data concerning the pharmacokinetic parameters of SN38 after IV Infusion of LE-SN38 at 2.5, 5 and 10 mg/m² to patients with advanced cancer in Strata A. Table 9 presents data concerning the pharmacokinetic parameters of SN38 after IV Infusion of LE-SN38 at 2.5 mg/m² to patients with advanced cancer in Strata B. Table 10 presents data concerning the mean maximum plasma concentration of SN38 (Cₘₐₓ) and area under the curve (AUC_{0-inf}) after LE-SN38 administration. Numbers reported for CPT-11 are drawn from published sources. FIG. 1 graphically presents values calculated for the mean (SD) plasma concentrations of SN38 after infusion of LE-SN38 at 2.5, 5 and 10 mg/m² to patients with advanced cancer in Strata A. FIG. 2 graphically presents data concerning the plasma concentrations of SN38 following infusion of LE-SN38 at 2.5 mg/m² to advanced cancer patients in Strata A and Strata B through the 4-day timepoint.

TABLE 8

| Dose | No. of | | Cₘₐₓ | | | AUC_{0-inf} | |
|---|---|---|---|---|---|---|---|
| mg/m² | Patients | Tₘₐₓ h | ng/mL | T_{1/2} h | Cl mL/min | ng * h/mL | Vss L |
| 2.5 | 3 | 1 | 68.0 | 12.2 | 790 | 143 | 174 |
| | | (0.43) | (45.9) | (8.19) | (480.6) | (87.6) | (50.1) |
| 5 | 6 | 1.06 | 95.9 | 13.3 | 775 | 220 | 178 |
| | | (0.55) | (33.9) | (10.9) | (311) | (104) | (85.7) |
| 10 | 3 | 1.25 | 177 | 18.9 | 753 | 396 | 429 |
| | | (0.43) | (63.9) | (14.7) | (128) | (107) | (495) |

TABLE 9

| | | | | | Cl | | |
|---|---|---|---|---|---|---|---|
| | | | Cₘₐₓ | | mL/ | AUC_{0-inf} | |
| Dose | No. of | Tₘₐₓ h | ng/ml | | min | ng * h/mL | Vss L |
| mg/m² | Patients | (SD) | (SD) | T_{1/2} h | (SD) | (SD) | (SD) |
| 2.5 | 4 | 1.02 | 40.6 | 7.59 | 869 | 90.8 | 205 |
| | | (0.64) | (19.7) | (3.20) | (328) | (26.9) | (144) |

TABLE 10

| | Dose of CPT-11 | SN38 Cₘₐₓ | SN38 AUC_{0-inf} |
|---|---|---|---|
| Clinical Study | or LESN38 | ng/mL | ng * hr/mL |
| (source) | (mg/m²) | (SD) | (SD) |
| DM111 | 50 | 21.0 (8.84) | 173 (92) |
| (Camptosar SBA) | | | |
| DM111 | 100 | 33.5 (13.3) | 581 (473) |
| (Camptosar SBA) | | | |
| DM111 | 165 | 49.0 (17.6) | 667 (484) |
| (Camptosar SBA) | | | |
| DM111 | 250 | 72.3 (40.9) | 876 (672) |
| (Camptosar SBA) | | | |
| DM111 | 350 | 139 | 1120 |
| (Camptosar SBA) | | | |
| M6475/0027 | 125 | 39.3 (4.7) | 450 (192) |
| (Camptosar SBA) | | | |
| M6475/0001 | 125 | 34.4 (15.0) | 459 (218) |
| (Camptosar SBA) | | | |
| LE-SN38-101 | 10 | 177 (63.9) | 396 (107) |
| (NeoPharm, Inc.) | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating viral infections comprising administering to a patient having a viral infection a composition comprising a complex comprising SN38 or a chemical in equilibrium with SN38 and a lipid to the patient in an amount sufficient to treat the viral infection within the patient.

2. A method of treating pancreatic cancer in a patient comprising administering to a patient having pancreatic cancer a complex comprising SN38 or a chemical in equilibrium with SN38 and a lipid to the patient in an amount sufficient to treat the cancer within the patient.

3. A method of treating a disease caused by proliferating eukaryotic cells in a patient homozygous for the wild-type UGTA1 allele or having at least one copy of the UGTA1*28 allele comprising administering to a patient having a disease caused by proliferating eukaryotic cells a complex comprising SN38 or a chemical in equilibrium with SN38 and a lipid to the patient in an amount sufficient to treat the disease caused by proliferating eukaryotic cells within the patient, wherein the patient has at least one copy of the UGTA1 allele.

4. A method of administering a composition comprising a composition selected from SN38 or a chemical in equilibrium with SN38 and a lipid to a human patient, wherein the composition is administered over a period of from about 30 to about 180 minutes.

5. A method of forming a lipid composition comprising an agent selected from SN38 or a chemical in equilibrium with SN38, involving forming a lipid phase and thereafter hydrating the lipid phase with a first aqueous solution including the compound so as to form lipid composition including the compound, and thereafter reducing the pH of the lipid composition, wherein the pH is reduced to less than 3.5.

6. The method of claim 5, wherein the pH is between about 1.5 and about 3.

7. The method of claim 6, wherein the pH is about 2.0.

8. The method of claim 5, wherein the lipid phase comprises cardiolipin.

9. The method of claim 8, wherein the cardiolipin is selected from a group consisting of natural cardiolipin, synthetic cardiolipin, and chemically modified cardiolipin.

10. The method of claim 8, wherein the lipid phase further comprises one or more lipids selected from a group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, ganglioside GM1 and polymer modified lipids, such as PEG modified lipids, and mixtures thereof.

11. The method of claim 10, wherein the phosphatidylglycerol is selected from a group consisting of dimyristoylphosphatidylglycerol, dioleoylphosphatidylglycerol, distearoylphosphatidylglycerol, dipalmitoylphosphatidylglycerol, diarachidonoylphosphatidylglycerol, or mixtures thereof.

12. The method of claim 10, wherein the phosphatidylcholine is selected from a group consisting of dimyristoylphosphatidylcholine, distearoylphosphatidylcholine, dioleoylphosphatidylcholine, dipalmitoylphosphatidylcholine, diarachidonoyl phosphatidylcholine, egg phosphatidylcholine, soy phosphatidylcholine, hydrogenated soy phosphatidylcholine, and mixtures thereof.

13. The method of claim 10, wherein the sterol is selected from a group consisting of cholesterol, polyethylene glycol derivatives of cholesterol, coprostanol, cholestanol, cholestane, cholesterol hemisuccinate, cholesterol sulfate, and mixtures thereof.

14. The method of claim 10, wherein the lipid phase consists of a phosphatidylcholine, a sterol, and a tocopherol.

15. The method of claim 5, wherein the lipid phase is formed in an organic solvent.

16. The method of claim 15, further involving evaporating the solvent.

17. The method of claim 16, wherein the evaporation occurs prior to reducing the pH of the composition.

18. The method of claim 5, wherein the first aqueous solution is a solubilizing agent that is employed to increase the solubility of SN38.

19. The method of claim 18, wherein the solubilizing agent has an alkaline pH.

20. The method of claim 19, wherein the solubilizing agent is selected from a group consisting of sodium carbonate, sodium bicarbonate, sodium hydroxide, sodium phosphate, ammonium acetate, sodium citrate, sodium hydroxide, calcium hydroxide, sodium biphosphate, sodium phosphate, Tris (hydroxy-methyl) aminomethane and sodium benzoate.

21. The method of claim 19, wherein the pH of the first aqueous solution is between about 7 and about 11.

22. The method of claim 21, wherein the pH of the first aqueous solution is between about 8 and about 10.

23. The method of claim 5, wherein the hydration step is performed with vigorous mixing.

24. The method of claim 5, further involving the addition of one or more agents to enhance the shelf-life of the SN38 composition.

25. The method of claim 24, wherein the agent comprises one or more sugars.

26. The method of claim 5, further involving filtering the lipid composition.

27. The method of claim 26, wherein the filtration occurs prior to reducing the pH of the composition.

28. The method of claim 26, wherein the filtration is through a filter of about 5 microns or less.

29. The method of claim 28, wherein the filtration is through a filter of about 1 micron or less.

30. The method of claim 29, wherein the filtration is through a filter of about 500 nm or less.

31. The method of claim 30, wherein the filtration is through a filter of about 200 nm or less.

32. The method of claim 31, wherein the filtration is through a filter of about 100 nm or less.

33. The method of claim 5, further involving dehydrating the lipid composition to form a dried lipid composition.

34. The method of claim 33, wherein the dehydrating occurs prior to reducing the pH of the composition.

35. The method of claim 33, wherein the dehydrating occurs after reducing the pH of the composition.

36. A liposomal composition comprising a compound selected from SN38 or a chemical in equilibrium with SN38 and one or more lipids, wherein said composition is produced in accordance with the method of claim 5.

37. The liposomal composition of claim 36, wherein 95 wt. % or more of the compound is in a complex with a portion of the lipid.

38. The composition of claim 36, wherein the compound is SN38.

39. The composition of claim 36, wherein the concentration of the compound in the composition is about 0.1 or more to about 20 mg/ml.

40. The composition of claim 36, wherein the concentration of the compound is about 0.01 or more to about 5 mg/ml.

41. The composition of claim 36, wherein the concentration of the compound is about 0.1 or more to about 4 mg/ml.

42. The composition of claim 36, wherein the concentration of the compound is about 0.5 or more to about 3 mg/ml.

43. The composition of claim 36, wherein the concentration of the compound is about 0.8 or more to about 2 mg/ml.

44. The composition of claim 36, wherein the concentration of the compound is about 1 or more to about 1.5 mg/ml.

45. The composition of claim 36, wherein the lipid comprises cardiolipin.

46. The composition of claim 45, wherein the cardiolipin is selected from a group consisting of natural cardiolipin, synthetic cardiolipin, and chemically modified cardiolipin.

47. The composition of claim 45, wherein the lipid further comprises one or more lipids selected from a group consisting of phosphatidylcholine, phosphatidylethanolamine, phosphatidylserine, phosphatidylglycerol, phosphatidic acid, phosphatidylinositol, sphingomyelin, sterol, tocopherol, fatty acid, ganglioside GM1 and polymer modified lipids, such as PEG modified lipids, and mixtures thereof.

48. The composition of claim 47, wherein the phosphatidylglycerol is selected from a group consisting of dimyristoylphosphatidylglycerol, dioleoylphosphatidylglycerol, distearoylphosphatidylglycerol, dipalmitoylphosphatidylglycerol, diarachidonoylphosphatidylglycerol, or mixtures thereof.

49. The composition of claim 47, wherein the phosphatidylcholine is selected from a group consisting of dimyristoylphosphatidylcholine, distearoylphosphatidylcholine, dioleoylphosphatidylcholine, dipalmitoylphosphatidylcholine, diarachidonoyl phosphatidylcholine, egg phosphatidylcholine, soy phosphatidylcholine, hydrogenated soy phosphatidylcholine, and mixtures thereof.

50. The composition of claim 47, wherein the sterol is selected from a group consisting of cholesterol, polyethylene glycol derivatives of cholesterol, coprostanol, cholestanol, cholestane, cholesterol hemisuccinate, cholesterol sulfate, and mixtures thereof.

51. The composition of claim 47, wherein the lipid consists of a phosphatidylcholine, a sterol, and a tocopherol.

52. The composition of claim 36, wherein the liposomes have a diameter of about 1 micron or less.

53. The composition of claim 52, wherein the liposomes have a diameter of about 200 nm or less.

54. The composition of claim 53, wherein the liposomes have a diameter of about 100 nm or less.

55. The composition of claim 36, further including a pharmaceutically acceptably excipient.

56. The composition of claim 36, further including a targeting agent.

57. The composition of claim 56, wherein the targeting agent is a protein.

58. The composition of claim 57, wherein the protein is selected from a group consisting of antibodies, antibody fragments, peptides, peptide hormones, receptor ligands, and mixtures thereof.

59. The composition of claim 56, wherein the targeting agent is a carbohydrate.
